(19) United States
(12) Patent Application Publication
(10) Pub. No.: US 2004/0005679 A1
(43) Pub. Date: Jan. 8, 2004

(54) Method of controlling zebra mussels

(76) Inventors: Matthew F. Ryan, Indianapolis, IN (US); Charles Clifton Chancey, Cedar Falls, IA (US)

Correspondence Address: Mr. Edward J. Timmer, Walnut Woods Centre, 5955 W. Main Street, Kalamazoo, MI, 49009, US

(21) Appl. No.: 10/437,270

(22) Filed: May 13, 2003

Related U.S. Application Data
(60) Provisional application No. 60/381,185, filed on May 16, 2002.

(51) Int. Cl. C12N 13/00

(52) U.S. Cl. 435/173.1

(57) ABSTRACT

A method for controlling infestation of zebra mussels in a manner that can be used to reduce fouling of water intake pipes and other water-conducting or carrying structures. The method involves using an oscillating magnetic dipole radiator to irradiate water of an infested site with an extremely low frequency (ELF) electromagnetic field to control zebra mussel infestation.

This application claims the benefits of provisional application Serial No. 60/381,185 filed May 16, 2002.

### FIELD OF THE INVENTION

The invention relates to a method of controlling zebra mussel infestation.

### BACKGROUND OF THE INVENTION

Since introduction into fresh waters of North America about 1985, the zebra mussel (Dreissena polymorpha) has spread throughout the waterways of the US and has extended its range to inland rivers and lakes including the Great Lakes. Zebra mussels can attach to a variety of solid substrates and can foul or block water intake systems as a consequence. Water intake systems are especially vulnerable to fouling or blockage if they draw from water sources that are infested.

### SUMMARY OF THE INVENTION

The present invention provides a method for controlling infestation of zebra mussels using an oscillating magnetic dipole radiator to reduce or eliminate zebra mussel infestation of water intake pipes and other water-conducting or water-containing compartments or structures.

In an illustrative embodiment of the invention, an oscillating magnetic dipole radiator (OMDR) generates an extremely low frequency (ELF) electromagnetic field having a frequency less than 300 Hertz to reduce or eliminate zebra mussel infestation at a site when positioned proximal to the site. The OMDR can comprise a wire coil disposed on a core. The wire coil is energized to generate an extremely low frequency (ELF) electromagnetic field having a frequency less than 300 Hertz to reduce or eliminate zebra mussel infestation at the site.

In another illustrative embodiment of the invention, a wire coil is wrapped around a water-carrying pipe or conduit about its exterior out of the water or about its interior in the water and is energized to generate the extremely low frequency electromagnetic field to reduce or eliminate zebra mussel infestation in the pipe or conduit.

### DESCRIPTION OF THE DRAWINGS

FIG. 1 is a schematic view of an oscillating magnetic dipole radiator immersed in water for practicing an illustrative method embodiment of the invention.

FIG. 2 is a schematic elevation view of an oscillating magnetic dipole radiator having a steel or iron rod and a wire coil wound thereon.

FIG. 2A is a schematic end elevation of the radiator of FIG. 2.

FIG. 3 is a schematic view of a water-carrying pipe or conduit.

FIG. 4 is a schematic elevation view of a wire coil that is wrapped about the water-carrying conduit of FIG. 3 and energized to generate an ELF electromagnetic field.

### DESCRIPTION OF THE INVENTION

The present invention provides a method for controlling infestation of zebra mussels (Dreissena polymorpha) in a non-chemical manner using an oscillating magnetic dipole radiator (OMDR) to reduce or eliminate infestation of water intake pipes or conduits and other water-conducting or water-containing compartments or structures. The invention can be practiced at water treatment facilities, electric power facilities, public utilities, canal locks, dams, and shipping vessels having bilges or other water-containing compartments, to reduce or eliminate zebra mussel infestation.

An embodiment of the invention involves irradiating water of an infested site with an extremely low frequency (ELF) electromagnetic field (e.g. a frequency less than 300 Hertz and an illustrative wavelength, lambda, on the order of 1×10⁶ meters or greater) using an oscillating magnetic dipole radiator (OMDR) 10 as an ELF electromagnetic field generator. The OMDR 10 is positioned in close enough proximity to the infested site to reduce or eliminate infestation of zebra mussels. The OMDR 10 can be immersed in the water or located out of the water yet in close enough proximity to the infested site to be effective to control zebra mussel infestation. For example, for purposes of illustration and not limitation, the OMDR 10 can be located within 10 meters or less of the infested site, or any other distance determined empirically or otherwise to be effective to reduce or eliminate zebra mussel infestation.

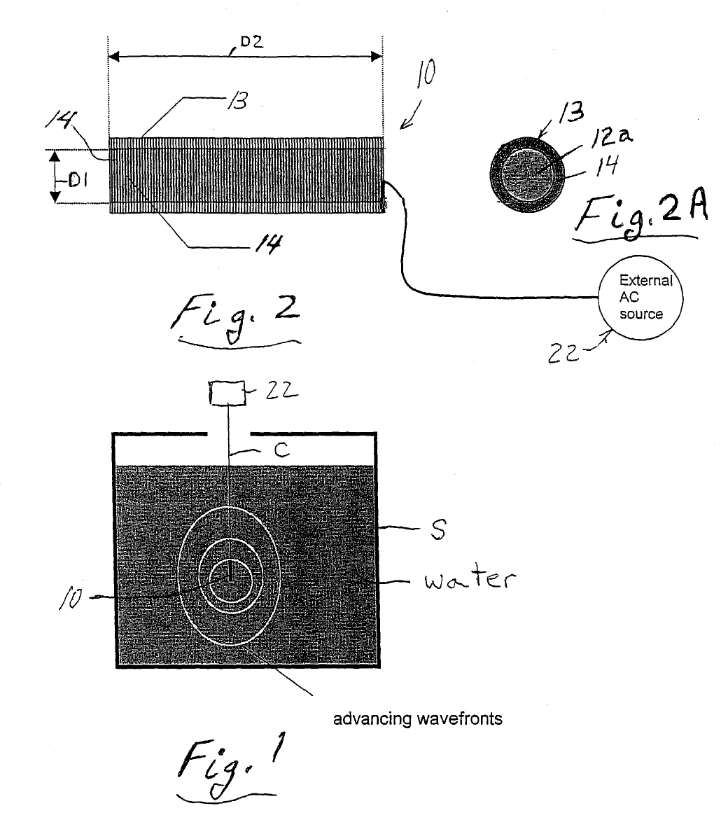

FIG. 1 illustrates an embodiment of the invention where the OMDR 10 comprises a core member 12a, such as a steel or iron cylindrical rod, having a wire coil 14 (e.g. magnet wire comprising copper wire coated with a polymer based dielectric coating) disposed on and wound around and along a length of core member 12a. In FIG. 1, the OMDR 10 is illustrated suspended under the water in a water-containing compartment or structure S, such for example in a ship's bilge compartment or in a lake or river water intake pipe prone to infestation of zebra mussels. The OMDR 10 is covered or coated with a polymer or other water-resistant coating or casing 13 to protect the components from corrosion and the like by the water. The OMDR 10 can be suspended in the compartment or structure S by a electrical power wire or cable C or affixed on a wall of the compartment or structure S.

The above-described OMDR 10 can be operated as an oscillating magnetic dipole radiator by passing a 60 Hertz to 240 Hertz AC signal through the wire coil 14 to generate an electromagnetic wave whose principal character is that of a time-varying bipolar magnetic signal. The electrical current and frequency of the AC signal can be modulated to increase efficacy of the OMDR. FIG. 1 provides a pictorial representation of the advancing wavefronts generated by the OMDR 10. Zebra mussels (not shown) on the interior walls of the compartment or structure S are bathed in the ELF electromagnetic field from OMDR 10 in a manner that has been found to disrupt the lifecycle of the zebra mussels and reduce or eliminate zebra mussel infestation. Although not wishing to be bound by any theory, the ELF electromagnetic radiation is believed to disrupt the ability of the zebra mussels to assimilate calcium and other ions and/or to cause loss of calcium and other ions from the zebra mussel shell and/or body. The OMDR may be energized continuously or in intermittent over time as necessary to control zebra mussel infestation.

For purposes of illustration and not limitation, the ELF electromagnetic field is generated by providing an electrical current of 0.5 ampere AC and 60 Hertz frequency through the wire coil 14 by connection of the wire coil via electrical power wire or cable C to external electrical power source 22 to this end. The wire coil can include any effective number of coil turns, such as for example only 1000 coils on the core rod 12a wrapped evenly along a length of the core rod 12a. The dimensions (diameter and length) of the core rod 12a can be selected as desired and effective for a given compartment or structure S. An exemplary rod diameter D1 can be in the range of 0.25 to 1.0 inch with a rod length, D2, of 5 times the diameter for purposes of illustration and not limitation.

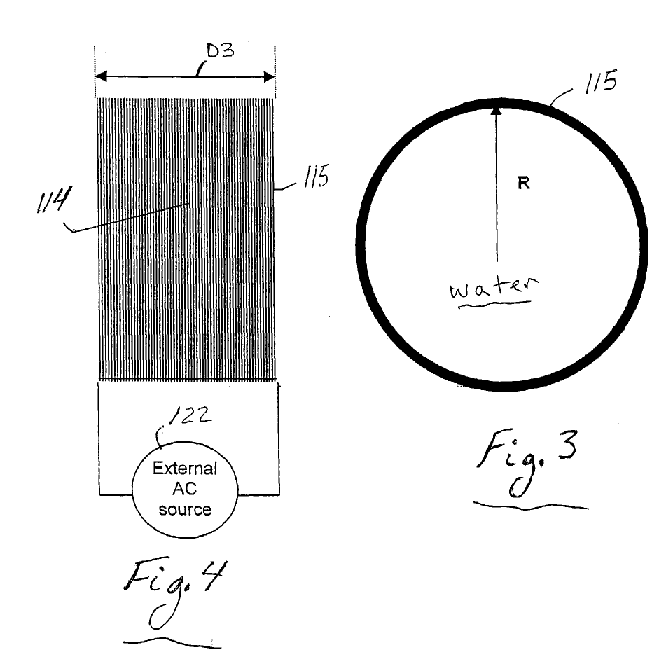

FIGS. 3-4 show another illustrative embodiment of the invention where a water intake pipe or other water-carrying conduit 115, FIG. 3, has a wire coil 114 (e.g. magnet wire) wrapped around the exterior and along a length of a water-intake pipe or conduit 115 through which water flows, FIG. 4, to function as an oscillating magnetic dipole radiator when the wire coil is energized appropriately. The water intake pipe or conduit 115 typically has a circular cross-section, FIG. 3, although any other pipe or conduit configuration can be used. The wire coil 114 can be covered or coated with a protective material or casing (not shown). The wire coil 114 is wrapped a linear distance D3 along a length of the pipe or conduit 115, the distance depending on the radius R of the conduit 115. For purposes of illustration and not limitation, 500 coils can wrapped per inch of radius R, and the distance D3 along the pipe or conduit 115 can be equal to 0.20R. For purposes of illustration and not limitation, the ELF electromagnetic field is generated by providing an electrical current of 0.5 ampere AC and 60 Hertz frequency through the coil wire 114 via connection to external AC electrical source 122 outside of the conduit 115. The ELF electromagnetic field from the OMDR baths the zebra mussels on the interior wall of the conduit 115 in an ELF electromagnetic field that has been found to disrupt the lifecycle of the zebra mussels and can reduce or eliminate zebra mussel infestation in closed water-containing compartments or structures and in flow-through, water-carrying conduits.

Although the invention has been described above with respect to certain embodiments, it is not so limited since modifications and changes can be made thereto within the scope of the invention as set forth in the appended claims.

We claim:

1. A method of controlling zebra mussels in water at an infested site, comprising positioning a magnetic dipole radiator proximal to said infested site and energizing said radiator in a manner to generate an extremely low frequency electromagnetic field having a frequency less than 300 Hertz effective to reduce or eliminate zebra mussel infestation.

2. The method of claim 1 wherein said radiator is immersed in the water at said site.

3. The method of claim 1 wherein said radiator is positioned out of the water at said site.

4. A method of controlling zebra mussels in water at an infested site, comprising positioning a magnetic dipole radiator comprising a wire coil disposed on a core in such close enough proximity to said infested site as to reduce or eliminate zebra mussel infestation when the wire coil is energized to generate an extremely low frequency electromagnetic field having a frequency less than 300 Hertz, and energizing said wire coil to generate said extremely low frequency electromagnetic field to reduce or eliminate zebra mussel infestation.

5. The method of claim 4 wherein said radiator is immersed in the water at said site.

6. The method of claim 4 wherein said radiator is positioned out of the water at said site.

7. The method of claim 4 wherein said wire coil is disposed on said core comprising steel or iron.

8. The method of claim 4 wherein said site is in a water-intake pipe.

9. The method of claim 4 wherein said site is in a closed water-containing compartment.

10. A method of controlling zebra mussels in water of a water intake pipe, comprising positioning a magnetic dipole radiator relative to the intake pipe and energizing said radiator to generate an extremely low frequency electromagnetic field having a frequency less than 300 Hertz to reduce or eliminate zebra mussel infestation in said intake pipe.

11. The method of claim 10 wherein a wire coil is positioned about and along a length of the exterior of the intake pipe, and the wire coil is energized to generate said electromagnetic field.

12. The method of claim 10 wherein said intake pipe is communicated to lake or river water.
